(19) United States
(12) Patent Application Publication
(10) Pub. No.: US 2019/0142748 A1
(43) Pub. Date: May 16, 2019

(54) FORMULATIONS OF CHLORAMBUCIL

(71) Applicant: Zhuhai Beihai Biotech Co., Ltd., Zhuhai (CN)

(72) Inventor: Qun Sun, Princeton, NJ (US)

(21) Appl. No.: 16/304,423

(22) PCT Filed: May 25, 2017
(86) PCT No.: PCT/US2017/034407

§ 371 Date: Nov. 26, 2018

Related U.S. Application Data
(60) Provisional application No. 62/506,067, filed on May 15, 2017.
(60) Provisional application No. 62/342,063, filed on May 26, 2016.

(51) Int. Cl. A61K 9/08; A61K 38/38; A61K 31/196; A61K 47/24; A61K 47/12; A61K 9/19

(52) U.S. Cl. CPC A61K 9/08; A61K 38/385; A61K 9/19; A61K 47/24; A61K 47/12; A61K 31/196

(57) ABSTRACT

This document relates to an aqueous composition comprising chlorambucil and human serum albumin, wherein the chlorambucil and the human serum albumin in the aqueous composition have a ratio by weight from about 1:10 to about 1:2000, wherein the aqueous composition comprises at least one water-miscible organic solvent. This document also relates to a solid composition comprising chlorambucil and human serum albumin. This document also relates to a liquid pharmaceutical composition comprising the solid composition comprising the chlorambucil and the human serum albumin, and a pharmaceutically acceptable carrier.

### TECHNICAL FIELD

This document relates to compositions and formulations for the treatment of proliferative diseases, and more particularly to compositions comprising chlorambucil.

### BACKGROUND

Chlorambucil is a nitrogen mustard that acts as a bifunctional alkylating agent and is used as a pharmaceutical agent. Chlorambucil is used primarily as an antineoplastic agent to treat cancer of the blood and lymphatic system, such as Hodgkin and non-Hodgkin lymphoma, chronic lymphocytic leukemia, and primary (Waldenstrom) macroglobulinemia. It is also used as a chemotherapeutic agent for Kaposi sarcoma and cancer of the breast, lung, cervix, ovary, and testis. Chlorambucil is an immunosuppressive agent that has been used to treat rheumatoid arthritis, systemic lupus erythematosus, acute and chronic glomerular nephritis, nephrotic syndrome, psoriasis, Wegener granulomatosis, chronic active hepatitis, and cold agglutinin disease.

Chlorambucil (LEUKERAN) is available in tablet form for oral administration. Chlorambucil interferes with DNA replication and induces cellular apoptosis via the accumulation of cytosolic p53 and subsequent activation of Bax, an apoptosis promoter. After single oral doses of 0.6 to 1.2 mg/kg, peak plasma chlorambucil levels (Cmax) are reached within 1 hour and the terminal elimination half-life (t½) of the parent drug is estimated at 1.5 hours. Chlorambucil is rapidly and completely (>70%) absorbed from the gastrointestinal tract. The absorption of chlorambucil is reduced when taken after food. In a study of ten patients, food intake increased the median Tmax by 2-fold and reduced the dose-adjusted Cmax and AUC values by 55% and 20%, respectively. Chlorambucil is extensively metabolized in the liver primarily to phenylacetic acid mustard, which has antineoplastic activity. Chlorambucil and its major metabolite undergo oxidative degradation to monohydroxy and dihydroxy derivatives. See LEUKERAN Prescribing Information.

Chlorambucil is assumed to be rapidly absorbed from the intestinal tract. However, overall bioavailability of Chlorambucil is not available due to the absence of an intravenous (IV) reference formulation. The instability of Chlorambucil in aqueous solution has prevented the development of a parenteral Chlorambucil formulation that could be used either for routine clinical administration or for pharmacological investigations. This lack of an IV formulation has prevented the development of optimal clinical administration schedules of Chlorambucil.

There exists a need for chlorambucil formulations that are suitable for parenteral administration such as intravascular administration. US patent application 2003/0082229 has reported a parenteral formulation of chlorambucil including a solvent, made of an alcohol and a lipid, such as soybean oil. However, there is further a need for chlorambucil formulations that are suitable for parenteral administration with no lipid, such as soybean oil and with no or less amount of alcohol.

### SUMMARY

Provided herein is an aqueous composition comprising chlorambucil and human serum albumin, wherein the chlorambucil and the human serum albumin in the aqueous composition have a ratio by weight from about 1:10 to about 1:2000. In some embodiments, the aqueous composition comprises at least one water-miscible organic solvent. In some embodiments, the chlorambucil and the human serum albumin in the aqueous composition have a ratio by weight from about 1:20 to about 1:1000. In some embodiments, the chlorambucil and the human serum albumin in the aqueous composition have a ratio by weight from about 1:30 to about 1:800. In some embodiments, the chlorambucil and the human serum albumin in the aqueous composition have a ratio by weight from about 1:30 to about 1:600. In some embodiments, the chlorambucil and the human serum albumin in the aqueous composition have a ratio by weight from about 1:30 to about 1:250. In some embodiments, the chlorambucil and the human serum albumin in the aqueous composition have a ratio by weight from about 1:20 to about 1:120. In some embodiments, the chlorambucil and the human serum albumin in the aqueous composition have a ratio by weight from about 1:30 to about 1:110. In some embodiments, the chlorambucil and the human serum albumin in the aqueous composition have a ratio by weight from about 1:30 to about 1:120. In some embodiments, the chlorambucil and the human serum albumin in the aqueous composition have a ratio by weight from about 1:30 to about 1:150. In some embodiments, the chlorambucil and the human serum albumin in the aqueous composition have a ratio by weight from about 1:40 to about 1:150. In some embodiments, the chlorambucil and the human serum albumin in the aqueous composition have a ratio by weight from about 1:40 to about 1:120. In some embodiments, the chlorambucil and the human serum albumin in the aqueous composition have a ratio by weight of about 1:10, about 1:20, about 1:30, about 1:40, about 1:50, about 1:60, about 1:70, about 1:80, about 1:90, about 1:100, about 1:110, or about 1:120. In some embodiments, the chlorambucil and the human serum albumin in the aqueous composition have a ratio by weight of about 1:30, about 1:40, about 1:50, about 1:60, about 1:80, or about 110.

In some embodiments, the human serum albumin is a native human serum albumin. In some embodiments, the human serum albumin is a recombinant human serum albumin. In some embodiments, the human serum albumin is essentially fatty acid free. In some embodiments, the human serum albumin can be an aqueous solution of human serum albumin comprising at least one stabilizer. In some embodiments, the human serum albumin can be an aqueous solution of human serum albumin comprising two stabilizers. In some embodiments, the stabilizers are N-acetyltryptophanate and caprylic acid or their sodium salts. In some embodiments, the stabilizer is N-acetyltryptophanate or its sodium salt. In some embodiments, the stabilizer is caprylic acid or its sodium salt.

In some embodiments, the water-miscible organic solvent in the aqueous composition is an alcohol. In some embodiments, the water-miscible organic solvent in the aqueous composition is methanol, ethanol, n-propanol, iso-propanol, n-butanol, tert-butanol, or mixtures thereof. In some embodiments, the water-miscible organic solvent in the aqueous composition is methanol. In some embodiments, the water-miscible organic solvent in the aqueous composition is ethanol.

In some embodiments, the aqueous composition comprising the chlorambucil and the human serum albumin is a clear aqueous solution. In some embodiments, the aqueous composition comprising the chlorambucil and the human serum albumin is a clear aqueous solution for at least 3 hours. In some embodiments, the aqueous composition comprising the chlorambucil and the human serum albumin is a clear aqueous solution for at least 6 hours.

Also, provided herein is a solid composition comprising chlorambucil and human serum albumin.

In some embodiments, the chlorambucil and the human serum albumin in the solid composition have a ratio by weight from about 1:20 to about 1:2000. In some embodiments, the chlorambucil and the human serum albumin in the solid composition have a ratio by weight from about 1:20 to about 1:1000. In some embodiments, the chlorambucil and the human serum albumin in the solid composition have a ratio by weight from about 1:30 to about 1:800. In some embodiments, the chlorambucil and the human serum albumin in the solid composition have a ratio by weight from about 1:30 to about 1:600. In some embodiments, the chlorambucil and the human serum albumin in the solid composition have a ratio by weight from about 1:30 to about 1:250. In some embodiments, the chlorambucil and the human serum albumin in the solid composition have a ratio by weight from about 1:40 to about 1:150. In some embodiments, the chlorambucil and the human serum albumin in the solid composition have a ratio by weight from about 1:40 to about 1:120. In some embodiments, the chlorambucil and the human serum albumin in the solid composition have a ratio by weight from about 1:20 to about 1:120. In some embodiments, the chlorambucil and the human serum albumin in the solid composition have a ratio by weight from about 1:30 to about 1:110. In some embodiments, the chlorambucil and the human serum albumin in the aqueous composition have a ratio by weight from about 1:30 to about 1:120. In some embodiments, the chlorambucil and the human serum albumin in the aqueous composition have a ratio by weight from about 1:30 to about 1:150.

In some embodiments, the chlorambucil and the human serum albumin in the solid composition have a ratio by weight of about 1:10, about 1:20, about 1:30, about 1:40, about 1:50, about 1:60, about 1:70, about 1:80, about 1:90, about 1:100, about 1:110, or about 1:120. In some embodiments, the chlorambucil and the human serum albumin in the solid composition have a ratio by weight of about 1:30, about 1:40, about 1:50, about 1:60, about 1:80, or about 110.

In some embodiments, the human serum albumin is a native human serum albumin. In some embodiments, the human serum albumin is a recombinant human serum albumin. In some embodiments, the human serum albumin is essentially fatty acid free.

In some embodiments, the human serum albumin is a powder produced by lyophilization of an aqueous solution of human serum albumin comprising at least one stabilizer. In some embodiments, the human serum albumin is a powder produced by lyophilization of an aqueous solution of human serum albumin comprising two stabilizers. In some embodiments, the stabilizers are N-acetyltryptophanate and caprylic acid or their sodium salts. In some embodiments, the stabilizer is N-acetyltryptophanate or its sodium salt. In some embodiments, the stabilizer is caprylic acid or its sodium salt.

In some embodiments, the solid composition is produced in a uniform manner by lyophilization. A skilled artisan would recognize other methods, such as rotary evaporation, that can also produce solid formulations.

In some embodiments, the solid composition is produced by lyophilization of the aqueous composition comprising the chlorambucil and the human serum albumin as described herein.

Also, provided herein is a liquid pharmaceutical composition comprising the solid composition comprising the chlorambucil and the human serum albumin as described herein, and a pharmaceutically acceptable carrier.

In some embodiments, the liquid pharmaceutical composition is a reconstituted solution, reconstituted from the solid composition comprising the chlorambucil and the human serum albumin as described herein.

In some embodiments, the liquid pharmaceutical composition is an aqueous solution.

In some embodiments, the liquid pharmaceutical composition is an aqueous reconstituted solution, reconstituted in a parenterally acceptable aqueous pharmaceutical diluent. In some embodiments, the liquid pharmaceutical composition is an aqueous reconstituted solution, reconstituted in an aqueous infusion fluid.

In some embodiments, the liquid pharmaceutical composition is an aqueous solution, wherein said composition contains not more than about 1% (area percent of chlorambucil) of the degradation product (I). In some embodiments, the liquid pharmaceutical composition is an aqueous solution, wherein said composition contains not more than about 2% (area percent of chlorambucil) of the degradation product (I). In some embodiments, the liquid pharmaceutical composition is an aqueous solution, wherein said composition contains not more than about 0.5% (area percent of chlorambucil) of the degradation product (I).

In some embodiments, the liquid pharmaceutical composition is free of solvent other than water. In some embodiments, the liquid pharmaceutical composition is substantially free of solvent other than water.

In some embodiments, the liquid pharmaceutical composition is an injectable pharmaceutical formulation.

In some embodiments, the injectable pharmaceutical formulation comprises at least one stabilizer. In some embodiments, the injectable pharmaceutical formulation comprises at least two stabilizers. In some embodiments, the injectable pharmaceutical formulation comprises two stabilizers. In some embodiments, the stabilizers are N-acetyltryptophanate and caprylic acid or their sodium salts. In some embodiments, the stabilizer is N-acetyltryptophanate or its sodium salt. In some embodiments, the stabilizer is caprylic acid or its sodium salt.

In some embodiments, the injectable pharmaceutical formulation is free of solvent other than water. In some embodiments, the injectable pharmaceutical formulation is substantially free of solvent other than water.

In some embodiments, the injectable pharmaceutical formulation is a reconstituted solution, reconstituted from the solid composition comprising the chlorambucil and the human serum albumin as described herein. In some embodiments, the injectable pharmaceutical formulation is a reconstituted solution, reconstituted in an aqueous infusion fluid. In some embodiments, the aqueous infusion fluid is 0.9% saline solution. In some embodiments, the aqueous infusion fluid is a dextrose solution.

In some embodiments, the injectable pharmaceutical formulation is a clear aqueous solution. In some embodiments, the injectable pharmaceutical formulation is a clear aqueous solution for at least 1 hour. In some embodiments, the injectable pharmaceutical formulation is a clear aqueous solution for at least 2 hours. In some embodiments, the injectable pharmaceutical formulation is a clear aqueous solution for at least 3 hours. In some embodiments, the injectable pharmaceutical formulation is a clear aqueous solution for at least 4 hours. In some embodiments, the injectable pharmaceutical formulation is a clear aqueous solution for at least 5 hours.

In some embodiments, the injectable pharmaceutical formulation is a clear aqueous solution for at least 1 hour at a temperature from about 0° C. to about 10° C. In some embodiments, the injectable pharmaceutical formulation is a clear aqueous solution for at least 2 hours at a temperature from about 0° C. to about 10° C. In some embodiments, the injectable pharmaceutical formulation is a clear aqueous solution for at least 3 hours at a temperature from about 0° C. to about 10° C. In some embodiments, the injectable pharmaceutical formulation is a clear aqueous solution for at least 6 hours at a temperature from about 0° C. to about 10° C. In some embodiments, the injectable pharmaceutical formulation is a clear aqueous solution for at least 8 hours at a temperature from about 0° C. to about 10° C. In some embodiments, the injectable pharmaceutical formulation is a clear aqueous solution for at least 24 hours at a temperature from about 0° C. to about 10° C.

In some embodiments, the injectable pharmaceutical formulation is free of a surfactant. In some embodiments, the surfactant is selected from CREMOPHOR® surfactants and Polysorbate 80.

In some embodiments, the injectable pharmaceutical formulation is substantially free of a surfactant. In some embodiments the surfactant is selected from CREMOPHOR® surfactants and Polysorbate 80.

Also, provided herein is a method of preparing a composition comprising chlorambucil and human serum albumin comprising the steps of:

(i) obtaining an organic solution of chlorambucil in polar water-miscible organic solvent;

(ii) obtaining a first aqueous solution comprising human serum albumin (HSA); and

(iii) mixing the organic solution comprising chlorambucil and the first aqueous solution comprising human serum albumin to obtain a second aqueous solution comprising the composition comprising chlorambucil and human serum albumin.

In some embodiments, the chlorambucil and the human serum albumin in the aqueous composition have a ratio by weight from about 1:30 to about 1:150.

In some embodiments, the human serum albumin is essentially fatty acid free.

In some embodiments, the amount of the polar water-miscible organic solvent in the organic solution is from about 0.033 mL to about 0.125 mL per 1 mg of chlorambucil.

In some embodiments, the polar water-miscible organic solvent is an alcohol selected from the group consisting of methanol, ethanol, isopropanol, n-butanol, and mixtures thereof.

In some embodiments, the polar water-miscible organic solvent is methanol.

In some embodiments, the amount of aqueous solvent in the first aqueous solution is from about 0.015 mL to about 0.033 mL per 1 mg of human serum albumin.

In some embodiments, the aqueous solvent is water.

In some embodiments, the mixing comprises adding the organic solution to the first aqueous solution.

In some embodiments, the mixing comprises adding the first aqueous solution to the organic solution.

In some embodiments, the mixing is carried out at about 0° C.

In some embodiments, the method further comprises removing the polar water-miscible organic solvent from the second aqueous solution to obtain a third aqueous solution comprising the composition comprising chlorambucil and human serum albumin.

In some embodiments, the method comprises removing aqueous solvent from the third aqueous solution to obtain the composition comprising chlorambucil and human serum albumin.

In some embodiments, the method further comprises removing the organic solvent and the aqueous solvent from the second aqueous solution to obtain the composition comprising chlorambucil and human serum albumin.

In some embodiments, the removing as carried out in vacuum.

In some embodiments, the removing is carried out by lyophilization.

Also, provided herein is a composition comprising chlorambucil and human serum albumin prepared by any one of the methods described herein.

In some embodiments, the composition is solid.

Also, provided herein is a liquid pharmaceutical composition comprising the composition prepared by any one of the methods described herein, and a pharmaceutically acceptable carrier.

In some embodiments, the pharmaceutically acceptable carrier is saline.

In some embodiments, liquid pharmaceutical composition is substantially free of solvent other than water.

In some embodiments the composition contains not more than about 1% of area percent of chlorambucil of the degradation product (I) as determined by LCMS.

In some embodiments the composition contains not more than about 0.5% of area percent of chlorambucil of the degradation product (I).

In some embodiments, the liquid pharmaceutical composition is a clear aqueous solution.

In some embodiments, the liquid pharmaceutical composition a clear aqueous solution for at least 1 hour.

In some embodiments, the liquid pharmaceutical composition is a clear aqueous solution for at least 2 hours.

In some embodiments, the liquid pharmaceutical composition is a clear aqueous solution for at least 6 hours at a temperature from about 0° C. to about 10° C.

In some embodiments, liquid pharmaceutical composition of is free from a surfactant selected from CREMOPHOR® surfactants and Polysorbate 80.

In some embodiments, the liquid pharmaceutical composition comprises a stabilizer selected from N-acetyltryptophanate and caprylic acid, or sodium salt thereof.

Also, provided herein is a unit dosage form for injection comprising the liquid pharmaceutical composition as described herein.

Also, provided herein is a method of treating a cancer, the method comprising the step of parenterally administering to a subject in need thereof of a therapeutically effective amount of a liquid pharmaceutical composition comprising the solid composition comprising the chlorambucil and the human serum albumin as described herein, and a pharmaceutically acceptable carrier.

In some embodiments, the cancer is selected from the group consisting of a chronic lymphocytic leukemia, a Non Hodgkin's lymphoma, a Hodgkin's lymphoma, and a Waldenstrom macroglobulinaemia. In some embodiments, the cancer is a chronic lymphocytic leukemia. In some embodiments, the cancer is a Non Hodgkin's lymphoma. In some embodiments, the cancer is a Hodgkin's lymphoma. In some embodiments, the cancer is a Waldenstrom macroglobulinaemia.

### DETAILED DESCRIPTION

Provided herein is an aqueous composition comprising chlorambucil and human serum albumin, wherein the chlorambucil and the human serum albumin in the aqueous composition have a ratio by weight from about 1:10 to about 1:2000. In some embodiments, the aqueous composition comprises at least one water-miscible organic solvent.

In some embodiments, the chlorambucil and the human serum albumin in the aqueous composition have a ratio by weight from about 1:20 to about 1:1000. In some embodiments, the chlorambucil and the human serum albumin in the aqueous composition have a ratio by weight from about 1:30 to about 1:800. In some embodiments, the chlorambucil and the human serum albumin in the aqueous composition have a ratio by weight from about 1:30 to about 1:600. In some embodiments, the chlorambucil and the human serum albumin in the aqueous composition have a ratio by weight from about 1:30 to about 1:250. In some embodiments, the chlorambucil and the human serum albumin in the aqueous composition have a ratio by weight from about 1:40 to about 1:150. In some embodiments, the chlorambucil and the human serum albumin in the aqueous composition have a ratio by weight from about 1:40 to about 1:120.

In some embodiments, the chlorambucil and the human serum albumin in the aqueous composition have a ratio by weight from about 1:20 to about 1:120. In some embodiments, the chlorambucil and the human serum albumin in the aqueous composition have a ratio by weight from about 1:30 to about 1:110. In some embodiments, the chlorambucil and the human serum albumin in the aqueous composition have a ratio by weight from about 1:30 to about 1:120. In some embodiments, the chlorambucil and the human serum albumin in the aqueous composition have a ratio by weight from about 1:30 to about 1:150.

In some embodiments, the chlorambucil and the human serum albumin in the aqueous composition have a ratio by weight of about 1:10, about 1:20, about 1:30, about 1:40, about 1:50, about 1:60, about 1:70, about 1:80, about 1:90, about 1:100, about 1:110, or about 1:120. In some embodiments, the chlorambucil and the human serum albumin in the aqueous composition have a ratio by weight of about 1:30, about 1:40, about 1:50, about 1:60, about 1:80, or about 110.

As used herein, the term “human serum albumin” refers to native and recombinant human serum albumin. Native human serum albumin and other plasma proteins can be precipitated from human plasma by varying the pH and adding ethanol, in what is known as the Cohn fractionation process (Cohn E J et al., J. Am. Chem. Soc. 1946; 68:459-475). By controlling the pH and ethanol content, semi-purified fractions of plasma proteins can be produced. One of the last proteins to precipitate in the Cohn process is native human serum albumin. After precipitation, a wet paste of crude native human serum albumin is obtained. Subsequent bioprocessing steps (purification, filtration, pasteurization, etc.) can be used to produce a purified, stabilized form of native human serum albumin for commercial use (Lin J J et al., Pharmaceutical Research 2000; 17:391-6). Recombinant human serum albumin is a highly purified animal-, virus-, and prion-free product as alternative to native human serum albumin, to which it is structurally equivalent (Bosse D et al., J. Clin. Pharmacol. 2005; 45:57-67). Recombinant human serum albumin has been produced by various hosts, both prokaryotic and eukaryotic (Chen Z et al., Biochimica et Biophysica Acta 2013; 1830:5515-5525). A fatty acid free human serum albumin can be prepared by treatment of human serum albumin with charcoal at low pH. Likewise, treatment of human serum albumin with charcoal at low pH can be used to remove fatty acids from human serum albumin (Chen R F, J. Biol. Chem. 1967; 242:173-181).

Human serum albumin (HSA) is a highly soluble globular protein of Mr 65K and consists of 585 amino acids. HSA is the most abundant protein in the plasma and accounts for 70-80% of the colloid osmotic pressure of human plasma. The amino acid sequence of HSA contains a total of 17 disulphide bridges, one free thiol (Cys 34), and a single tryptophan (Trp 214). Intravenous use of HSA solution has been indicated for the prevention and treatment of hypovolumic shock (see, e.g., Tullis, JAMA, 237, 355-360, 460-463, (1977) and Houser et al., Surgery, Gynecology and Obstetrics, 150, 811-816 (1980)) and in conjunction with exchange transfusion in the treatment of neonatal hyperbilirubinemia (see, e.g., Finlayson, Seminars in Thrombosis and Hemostasis, 6, 85-120, (1980)).

Human serum albumin (HSA) has multiple hydrophobic binding sites (a total of seven for medium and long-chain fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Two high affinity binding sites have been proposed in subdomains IIA and IIIA of HSA, which are highly elongated hydrophobic pockets with charged lysine and arginine residues near the surface which function as attachment points for polar ligand features (see, e.g., Fehske et al., Biochem. Pharmcol., 30, 687-92 (1981), Vorum, Dan. Med. Bull., 46, 379-99 (1999), Kragh-Hansen, Dan. Med Bull., 1441, 131-40 (1990), Curry et al., Nat. Struct. Biol., 5, 827-35 (1998), Sugio et al., Protein. Eng., 12, 439-46 (1999), He et al., Nature, 358, 209-15 (1992), and Carter et al., Adv. Protein. Chem., 45, 153-203 (1994)).

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free. In some embodiments, the human serum albumin contains no more than two moles of fatty acids bound to one mole of human serum albumin. In some embodiments, the human serum albumin contains no more than one mole of fatty acids bound to one mole of human serum albumin. In some embodiments, human serum albumin contains no more than 0.5 moles of fatty acids bound to one mole of human serum albumin. In some embodiments, the human serum albumin contains no more than 0.1 moles of fatty acids bound to one mole of human serum albumin. In some embodiments, the human serum albumin contains no more than 0.05 moles of fatty acids bound to one mole of human serum albumin. In some embodiments, the human serum albumin contains no more than 0.01 moles of fatty acids bound to one mole of human serum albumin. In some embodiments, the human serum albumin contains no more than 0.001 moles of fatty acids bound to one mole of human serum albumin. In some embodiments, the human serum albumin contains no more than 0.0005 moles of fatty acids bound to one mole of human serum albumin. In some embodiments, the human serum albumin contains no more than 0.0001 moles of fatty acids bound to one mole of human serum albumin.

Chlorambucil is extensively bound to plasma and tissue proteins. In vitro, chlorambucil is 99% bound to plasma proteins, specifically albumin. See LEUKERAN Prescribing Information.

As used herein, the term “essentially fatty acid free” refers to proteins (e.g. serum albumin) that contain less than about 0.02% fatty acid by weight. For example, human serum albumin that is essentially fatty acid free can contain less than 0.02% fatty acid by weight.

As used herein, the term “fatty acids” refers to non-esterified fatty acids (e.g. linoleic acid, α-linoleic acid, γ-linoleic acid).

Solutions of human serum albumin for infusion are commercially available. Those solutions must be supplemented with stabilizers to allow pasteurization and storage, to avoid the spontaneous polymerization of the albumin. Usually, N-acetyltryptophanate and caprylic acid or their sodium salts are used in alone or in combination.

In some embodiments, the human serum albumin is a commercially available solution of human serum albumin USP for infusion. In some embodiments, the solution of human serum albumin USP for infusion is 5% solution of human serum albumin USP. In some embodiments, the solution of human serum albumin USP for infusion is 20% solution of human serum albumin USP. In some embodiments, the solution of human serum albumin USP for infusion is 25% solution of human serum albumin USP.

In some embodiments, the human serum albumin can be an aqueous solution of human serum albumin comprising at least one stabilizer. In some embodiments, the human serum albumin can be an aqueous solution of human serum albumin comprising two stabilizers. In some embodiments, the stabilizers are N-acetyltryptophanate and caprylic acid or their sodium salts. In some embodiments, the stabilizer is N-acetyltryptophanate or its sodium salt. In some embodiments, the stabilizer is caprylic acid or its sodium salt.

As used herein the term “chlorambucil” is a compound that has the CAS No. 305-03-3 and the following chemical structure:

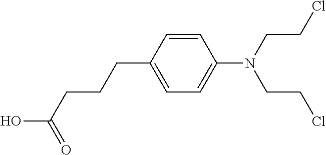 [SMILES: O=C(O)CCCc1ccc(N(CCCl)CCCl)cc1]

Chlorambucil is a white or almost white, crystalline powder, practically insoluble in water, freely soluble in acetone and in alcohol.

Chlorambucil is a nitrogen mustard alkylating agent and can be given orally. Chlorambucil produces its anti-cancer effects by interfering with DNA replication and damaging the DNA in a cell. The DNA damage induces cell cycle arrest and cellular apoptosis via the accumulation of cytosolic p53 and subsequent activation of Bax, an apoptosis promoter.

Chlorambucil has been mainly used in the treatment of chronic lymphocytic leukemia, Hodgkin and non-Hodgkin lymphoma, and primary (Waldenstrom) macroglobulinemia.

In some embodiments, the chlorambucil can be a pharmaceutically acceptable salt of chlorambucil.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively. In some embodiments, pharmaceutically acceptable salts may be preferred over the respective free base or free acid because such salts impart greater stability or solubility to the molecule thereby facilitating formulation into a dosage form. Basic compounds are generally capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate),napthalene-2-sulfonate, ethanedisulfonate, hydrogen bisulfide, bitartrate, gluconate, glucuronate, para-bromophenylsulfonate, carbonate, pyrosulfate, sulfite, bisulfate, monohydrogen phosphate, dihydrogen phosphate, metaphosphate, pyrophosphate, chloride, bromide, iodide, decanoate, caprylate, caprate, propiolate, suberate, sebacate, butyne-1,4-dioate, hexyne-1,6-dioate, terephthalate, sulfonate, xylenesulfonate, phenylpropionate, phenylbutyrate, β-hydroxybutyrate, glycolate, propanesulfonate, naphthalene-l-sulfonate, naphthalene-2-sulfonate and 2,5-dihydroxybenzoate. Suitable bases include pharmaceutically acceptable inorganic bases and pharmaceutically acceptable organic bases. Representative pharmaceutically acceptable base addition salts include hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH-(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the chlorambucil can be a hydrochloride salt of chlorambucil.

In some embodiments, the water-miscible organic solvent in the aqueous composition is an alcohol. In some embodiments, the water-miscible organic solvent in the aqueous composition is methanol, ethanol, n-propanol, iso-propanol, n-butanol, tert-butanol, or mixtures thereof. In some embodiments, the water-miscible organic solvent in the aqueous composition is methanol. In some embodiments, the water-miscible organic solvent in the aqueous composition is ethanol.

In some embodiments, the water-miscible organic solvent in the aqueous composition is polyethylene glycol 300, polyethylene glycol 400, ethanol, methanol, propylene glycol, glycerin, N-methyl-2-pyrrolidone, dimethylacetamide, and dimethylsulfoxide, or mixtures thereof. In some embodiments, the water-miscible organic solvent is the mixture of methanol and ethanol.

In some embodiments, the aqueous composition comprising chlorambucil and human serum albumin is a clear aqueous solution.

As used herein, the term “clear aqueous solution” refers to a solution containing chlorambucil and human serum albumin in an aqueous solution that is transparent upon visual observation and essentially free of visible particles or precipitation of undissolved chlorambucil.

The term “essentially free of visible particles or precipitation of undissolved chlorambucil” can be assessed as follows: after a clear aqueous solution is filtered with a 0.22 micron filter, the amount of chlorambucil in the filtered aqueous solution is at least 95% of the total amount of chlorambucil in the aqueous solution before filtration. The total amount of chlorambucil in the aqueous solution before filtration includes the particles or precipitation of undissolved chlorambucil in the aqueous solution or with the aqueous solution. The amount of the chlorambucil in an aqueous solution can be measured by the methods using HPLC. The methods of measuring the amount of the chlorambucil in an aqueous solution are illustrated in the experimental examples described herein. The methods are commonly understood by one of ordinary skill in the art to which this disclosure belongs.

When visually observed, for example, the term “clear aqueous solution” excludes a milky aqueous solution. Further, the term “clear aqueous solution” excludes a cloudy or hazy aqueous solution.

As used herein, the term “micron” refers to a unit of measure of one one-thousandth of a millimeter.

In some embodiments, the aqueous composition comprising chlorambucil and human serum albumin has pH value from about 5 to about 8. In some embodiments, the aqueous composition comprising chlorambucil and human serum albumin has pH value from about 5.5 to about 7.8. In some embodiments, the aqueous composition comprising chlorambucil and human serum albumin has pH value from about 6 to about 7.5. In some embodiments, the aqueous composition comprising chlorambucil and human serum albumin has pH value from about 6.5 to about 7.5. In some embodiments, the aqueous composition comprising chlorambucil and human serum albumin has pH value from about 6 to about 6.5. In some embodiments, the aqueous composition comprising chlorambucil and human serum albumin has pH value from about 6.5 to about 7. In some embodiments, the aqueous composition comprising chlorambucil and human serum albumin has pH value from about 7 to about 7.5. In some embodiments, the aqueous composition comprising chlorambucil and human serum albumin has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the aqueous composition comprising chlorambucil and human serum albumin is substantially free of solvent other than water. In some embodiments, the aqueous composition comprising chlorambucil and human serum albumin is free of solvent other than water.

In some embodiments, the aqueous composition comprising chlorambucil and human serum albumin is a clear aqueous solution, wherein the aqueous solution has pH value from about 5 to about 8, and wherein the aqueous solution is substantially free of solvent other than water. In some embodiments, the aqueous composition comprising chlorambucil and human serum albumin is a clear aqueous solution, wherein the aqueous formulation has pH value from about 6 to about 7.5, and wherein the aqueous formulation is substantially free of solvent other than water.

In some embodiments, the aqueous composition comprising chlorambucil and human serum albumin is a clear aqueous solution for at least 1 hours. In some embodiments, the aqueous composition comprising chlorambucil and human serum albumin is a clear aqueous solution for at least 2 hours. In some embodiments, the aqueous composition comprising chlorambucil and human serum albumin is a clear aqueous solution for at least 3 hours. In some embodiments, the aqueous composition comprising chlorambucil and human serum albumin is a clear aqueous solution for at least 6 hours. In some embodiments, the aqueous composition comprising chlorambucil and human serum albumin is a clear aqueous solution for a period of time selected from 1 hour. 2 hours, 3 hours, 4 hours, 5 hours, 6 hours, 7 hours, 8 hours, 12 hours, and 24 hours. In some embodiments, the aqueous composition comprising chlorambucil and human serum albumin is a clear aqueous solution for at least 24 hours.

Also, provided herein is a solid composition comprising chlorambucil and human serum albumin. In some embodiments, the chlorambucil and the human serum albumin in the solid composition are bound non-covalently. In some embodiments, the solid composition comprises a non-covalently bond complex comprising chlorambucil and the human serum albumin.

As used herein, the term “non-covalent” refers to an interaction between two or more components, wherein the bonds between the components are non-covalent bonds (i.e., no atom of one component shares a pair of electrons with an atom of another component; e.g., weak bonds such as hydrogen bonds, electrostatic effects, π-effects, hydrophobic effects and Van der Waals forces). Further, human serum albumin (HSA) has multiple hydrophobic binding sites (a total of seven for medium and long-chain fatty acids, an endogenous ligand of HSA) and binds a diverse set of drugs, especially neutral and negatively charged hydrophobic compounds (Goodman et al., The Pharmacological Basis of Therapeutics, 9th ed, McGraw-Hill New York (1996)). Additionally, after the drug molecule binds to HSA, the drug molecule and HSA form a non-covalently bound drug and protein complex through the binding sites of HSA. This concept is commonly understood by one of ordinary skill in the art to which this disclosure belongs. One example of a non-covalently bound complex is a non-covalently bound complex of HSA and fatty acids, in which the fatty acids bind to HSA through HSA's multiple binding sites.

In some embodiments, the non-covalent interaction between chlorambucil and human serum albumin comprises hydrogen bonding. In some embodiments, the non-covalent interaction between chlorambucil and human serum albumin comprises electrostatic interaction. In some embodiments, the non-covalent interaction between chlorambucil and human serum albumin comprises hydrophobic interaction. In some embodiments, the non-covalent interaction between chlorambucil and human serum albumin comprises Van der Waals forces. In some embodiments, the non-covalent interaction between chlorambucil and human serum albumin comprises hydrogen bonding, electrostatic interaction, hydrophobic interaction, and Van der Waals forces.

In some embodiments, the chlorambucil and the human serum albumin in the solid composition have a ratio by weight from about 1:20 to about 1:2000. In some embodiments, the chlorambucil and the human serum albumin in the solid composition have a ratio by weight from about 1:20 to about 1:1000. In some embodiments, the chlorambucil and the human serum albumin in the solid composition have a ratio by weight from about 1:30 to about 1:800. In some embodiments, the chlorambucil and the human serum albumin in the solid composition have a ratio by weight from about 1:30 to about 1:600. In some embodiments, the chlorambucil and the human serum albumin in the solid composition have a ratio by weight from about 1:30 to about 1:250. In some embodiments, the chlorambucil and the human serum albumin in the solid composition have a ratio by weight from about 1:40 to about 1:150. In some embodiments, the chlorambucil and the human serum albumin in the solid composition have a ratio by weight from about 1:40 to about 1:120. In some embodiments, the chlorambucil and the human serum albumin in the solid composition have a ratio by weight from about 1:20 to about 1:120. In some embodiments, the chlorambucil and the human serum albumin in the solid composition have a ratio by weight from about 1:30 to about 1:110. In some embodiments, the chlorambucil and the human serum albumin in the aqueous composition have a ratio by weight from about 1:30 to about 1:120. In some embodiments, the chlorambucil and the human serum albumin in the aqueous composition have a ratio by weight from about 1:30 to about 1:150.

In some embodiments, the chlorambucil and the human serum albumin in the solid composition have a ratio by weight of about 1:10, about 1:20, about 1:30, about 1:40, about 1:50, about 1:60, about 1:70, about 1:80, about 1:90, about 1:100, about 1:110, or about 1:120. In some embodiments, the chlorambucil and the human serum albumin in the solid composition have a ratio by weight of about 1:30, about 1:40, about 1:50, about 1:60, about 1:80, or about 110.

In some embodiments, the human serum albumin is a native human serum albumin. In some embodiments, the human serum albumin is a recombinant human serum albumin. In some embodiments, the human serum albumin is a fatty acid free human serum albumin. In some embodiments, the human serum albumin is essentially fatty acid free. In some embodiments, the human serum albumin contains no more than two moles of fatty acids bound to one mole of human serum albumin. In some embodiments, the human serum albumin contains no more than one mole of fatty acids bound to one mole of human serum albumin. In some embodiments, human serum albumin contains no more than 0.5 moles of fatty acids bound to one mole of human serum albumin. In some embodiments, the human serum albumin contains no more than 0.1 moles of fatty acids bound to one mole of human serum albumin. In some embodiments, the human serum albumin contains no more than 0.05 moles of fatty acids bound to one mole of human serum albumin. In some embodiments, the human serum albumin contains no more than 0.01 moles of fatty acids bound to one mole of human serum albumin. In some embodiments, the human serum albumin contains no more than 0.001 moles of fatty acids bound to one mole of human serum albumin. In some embodiments, the human serum albumin contains no more than 0.0005 moles of fatty acids bound to one mole of human serum albumin. In some embodiments, the human serum albumin contains no more than 0.0001 moles of fatty acids bound to one mole of human serum albumin.

In some embodiments, the human serum albumin is a powder produced by lyophilization of an aqueous solution of human serum albumin comprising at least one stabilizer. In some embodiments, the human serum albumin is a powder produced by lyophilization of an aqueous solution of human serum albumin comprising two stabilizers. In some embodiments, the stabilizers are N-acetyltryptophanate and caprylic acid or their sodium salts. In some embodiments, the stabilizer is N-acetyltryptophanate or its sodium salt. In some embodiments, the stabilizer is caprylic acid or its sodium salt.

In some embodiments, the human serum albumin is a powder produced by lyophilization of a commercially available solution of human serum albumin USP for infusion. In some embodiments, the solution of human serum albumin USP for infusion is 5% solution of human serum albumin USP. In some embodiments, the solution of human serum albumin USP for infusion is 20% solution of human serum albumin USP. In some embodiments, the solution of human serum albumin USP for infusion is 25% solution of human serum albumin USP.

In some embodiments, the solid composition is produced in a uniform manner by lyophilization. A skilled artisan would recognize other methods, such as rotary evaporation, that can also produce solid formulations.

In some embodiments, the solid composition is produced by lyophilization of the aqueous composition comprising the chlorambucil and the human serum albumin as described herein.

Also, provided herein is a liquid pharmaceutical composition comprising the solid composition comprising the chlorambucil and the human serum albumin as described herein, and a pharmaceutically acceptable carrier.

As used herein, the term “pharmaceutically acceptable carrier” is refers to any carrier useful to solubilize and deliver an agent to a subject. A desirable pharmaceutically acceptable carrier is saline. Other pharmaceutically acceptable carrier and their formulation are known to one skilled in the art and described, for example, in Remington's Pharmaceutical Sciences. (20th edition), ed. A. Gennaro, 2003, Lippincon Williams & Wilkins. In some embodiments, the carrier may contain components such as, for example, dextrose, glucose, serum proteins (other than HSA), buffer substances such as phosphates, glycine, sorbic acid, potassium sorbate, salts or electrolytes, such as protamine sulfate, disodium hydrogen phosphate, potassium hydrogen phosphate, sodium chloride, zinc salts, and cellulose-based substances. In some embodiments, the carrier may contain components such as contain anti-oxidants, buffers, bacteriostats and solutes which render the formulation isotonic with the blood of the intended recipient.

In some embodiments, the liquid pharmaceutical composition of the present disclosure may be administered by a syringe or a catheter, or any other means generally known in the art for the delivery of a pharmaceutical agent by injection to the subject in need thereof. The delivery means will vary, as recognized by those skilled in the art, depending on the diseases and conditions treated, the severity of the disease, the sex, age and general health condition of the subject, excipient usage, the possibility of co-usage with other therapeutic treatments such as use of other agents as described herein and the judgment of the treating physician.

In some embodiments, the liquid pharmaceutical composition is a reconstituted solution, reconstituted from the solid composition comprising the chlorambucil and the human serum albumin as described herein.

In some embodiments, the liquid pharmaceutical composition is an aqueous solution.

As used herein, the term “aqueous solution” refers to a solution, wherein at least one solvent is water and the weight % of water in the mixture of solvents is at least 50%, at least 60%, at least 70% or at least 90%. In some embodiments, aqueous solution is a solution in which water is the only solvent.

As used herein, the term “aqueous solvent” refer to a liquid comprising at least 50%, at least 60%, at least 70%, at least 90% or at least 95% water. In some embodiments, aqueous solvent is water.

In some embodiments, the liquid pharmaceutical composition is an aqueous reconstituted solution, reconstituted in a parenterally acceptable aqueous pharmaceutical diluent. In some embodiments, the liquid pharmaceutical composition is an aqueous reconstituted solution, reconstituted in an aqueous infusion fluid.

In some embodiments, the liquid pharmaceutical composition further comprises at least one anti-cancer drug (e.g., any one of the anti-cancer drugs as described herein).

Chlorambucil is degraded rapidly to its degradation product (I), 4-[p-(2-chloroethyl-2-hydroxyethylamino) phenyllbutyric acid, in an aqueous solution (Ehrsson H et al., J. Pharmaceutical Sciences 1980; 69:1091-1094). The degradation product (I) is further degraded to the degradation product (II), 4-(4-(bis(2-hydroxyethyl)amino)phenyl) butanoic acid in an aqueous solution. Both the degradation product (I) and the degradation product (II) are inactive metabolites of chlorambucil.

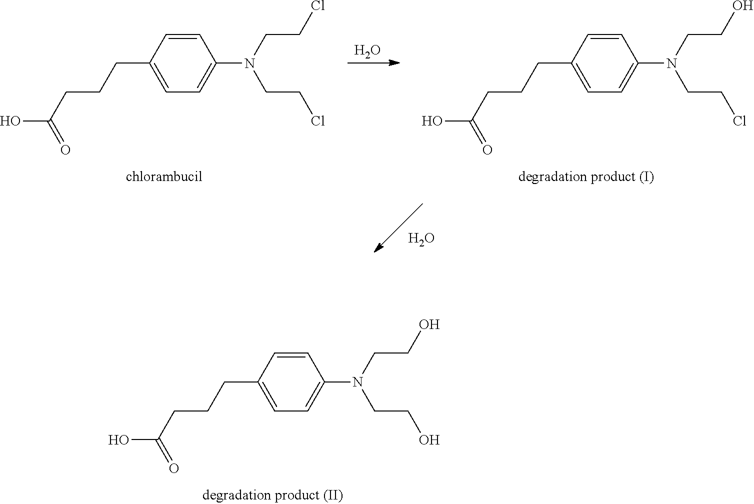 [SMILES: O=C(O)CCCc1ccc(N(CCCl)CCCl)cc1 ; O ; O=C(O)CCCc1ccc(N(CCO)CCCl)cc1 ; O ; O=C(O)CCCc1ccc(N(CCO)CCO)cc1]

In some embodiments, the liquid pharmaceutical composition is an aqueous solution, wherein said composition contains not more than about 1% (area percent of chlorambucil) of the degradation product (I). The area percent of chlorambucil and the area percent of the degradation product (I) can be measured by the methods using HPLC. The methods of measuring the area percent of chlorambucil and the area percent of the degradation product (I) are illustrated in the experimental examples described herein. The methods are commonly understood by one of ordinary skill in the art to which this disclosure belongs.

In some embodiments, the liquid pharmaceutical composition is an aqueous solution, wherein said composition contains not more than about 2% (area percent of chlorambucil) of the degradation product (I).

In some embodiments, the liquid pharmaceutical composition is an aqueous solution, wherein said composition contains not more than about 0.5% (area percent of chlorambucil) of the degradation product (I).

In some embodiments, the liquid pharmaceutical composition is an aqueous solution, wherein said composition contains not more than about 0.3% (area percent of chlorambucil) of the degradation product (I).

In some embodiments, the liquid pharmaceutical composition is an aqueous solution, wherein said composition contains not more than about 0.1% (area percent of chlorambucil) of the degradation product (I).

In some embodiments, the liquid pharmaceutical composition is free of solvent other than water. In some embodiments, the liquid pharmaceutical composition is substantially free of solvent other than water.

As used herein, “substantially free of solvent,” in reference to an aqueous solution, refers to an aqueous solution that contains less than 0.5%, by weight, of any non-water solvent. In some embodiments, the aqueous solution contains less than 0.1%, by weight, of any non-water solvent. In some embodiments, the aqueous solution contains less than 0.05%, by weight, of any non-water solvent.

In some embodiments, the liquid pharmaceutical composition has pH value from about 5 to about 8. In some embodiments, the liquid pharmaceutical composition has pH value from about 5.5 to about 7.8. In some embodiments, the liquid pharmaceutical composition has pH value from about 6 to about 7.5. In some embodiments, the liquid pharmaceutical composition has pH value from about 6.5 to about 7.5. In some embodiments, the liquid pharmaceutical composition has pH value from about 6 to about 6.5. In some embodiments, the liquid pharmaceutical composition has pH value from about 6.5 to about 7. In some embodiments, the liquid pharmaceutical composition has pH value from about 7 to about 7.5. In some embodiments, the liquid pharmaceutical composition has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the liquid pharmaceutical composition is substantially free of solvent other than water. In some embodiments, the liquid pharmaceutical composition is free of solvent other than water.

In some embodiments, the liquid pharmaceutical composition is a clear aqueous solution, wherein the aqueous solution has pH value from about 5 to about 8, and wherein the aqueous solution is substantially free of solvent other than water. In some embodiments, the liquid pharmaceutical composition is a clear aqueous solution, wherein the aqueous solution has pH value from about 5 to about 8, and wherein the aqueous solution is free of solvent other than water. In some embodiments, the liquid pharmaceutical composition is a clear aqueous solution, wherein the aqueous solution has pH value from about 6 to about 7.5, and wherein the aqueous solution is substantially free of solvent other than water. In some embodiments, the liquid pharmaceutical composition is a clear aqueous solution, wherein the aqueous solution has pH value from about 6 to about 7.5, and wherein the aqueous solution is free of solvent other than water.

In some embodiments, the liquid pharmaceutical composition is an injectable pharmaceutical formulation.

In some embodiments, the injectable pharmaceutical formulation comprises at least one stabilizer. In some embodiments, the injectable pharmaceutical formulation two stabilizers. In some embodiments, the stabilizers are N-acetyltryptophanate and caprylic acid or their sodium salts. In some embodiments, the stabilizer is N-acetyltryptophanate or its sodium salt. In some embodiments, the stabilizer is caprylic acid or its sodium salt.

In some embodiments, the injectable pharmaceutical formulation is free of solvent other than water. In some embodiments, the injectable pharmaceutical formulation is substantially free of solvent other than water.

In some embodiments, the injectable pharmaceutical formulation is a reconstituted solution, reconstituted from the solid composition comprising the chlorambucil and the human serum albumin as described herein. In some embodiments, the injectable pharmaceutical formulation is a reconstituted solution, reconstituted in an aqueous infusion fluid. In some embodiments, the aqueous infusion fluid is 0.9% saline solution. In some embodiments, the aqueous infusion fluid is a dextrose solution.

In some embodiments, the injectable pharmaceutical formulation has pH value from about 5 to about 8. In some embodiments, the injectable pharmaceutical formulation has pH value from about 5.5 to about 7.8. In some embodiments, the injectable pharmaceutical formulation has pH value from about 6 to about 7.5. In some embodiments, the injectable pharmaceutical formulation has pH value from about 6.5 to about 7.5. In some embodiments, the injectable pharmaceutical formulation has pH value from about 6 to about 6.5. In some embodiments, the injectable pharmaceutical formulation has pH value from about 6.5 to about 7. In some embodiments, the injectable pharmaceutical formulation has pH value from about 7 to about 7.5. In some embodiments, the injectable pharmaceutical formulation has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the injectable pharmaceutical formulation is substantially free of solvent other than water. In some embodiments, the injectable pharmaceutical formulation is free of solvent other than water.

In some embodiments, the injectable pharmaceutical formulation is a clear aqueous solution, wherein the aqueous solution has pH value from about 5 to about 8, and wherein the aqueous solution is substantially free of solvent other than water. In some embodiments, the injectable pharmaceutical formulation is a clear aqueous solution, wherein the aqueous solution has pH value from about 6 to about 7.5, and wherein the aqueous solution is substantially free of solvent other than water.

In some embodiments, the injectable pharmaceutical formulation is a clear aqueous solution. In some embodiments, the injectable pharmaceutical formulation is a clear aqueous solution for at least 1 hour. In some embodiments, the injectable pharmaceutical formulation is a clear aqueous solution for at least 2 hours. In some embodiments, the injectable pharmaceutical formulation is a clear aqueous solution for at least 3 hours. In some embodiments, the injectable pharmaceutical formulation is a clear aqueous solution for at least 4 hours. In some embodiments, the injectable pharmaceutical formulation is a clear aqueous solution for at least 5 hours. In some embodiments, the injectable pharmaceutical formulation is a clear aqueous solution for a period of time selected from 1 hour, 2 hours, 3 hours, 4 hours, 5 hours, 6 hours, 7 hours, 8 hours, 12 hours, and 24 hours.

In some embodiments, the injectable pharmaceutical formulation is a clear aqueous solution for at least 1 hour at a temperature from about 0° C. to about 10° C. In some embodiments, the injectable pharmaceutical formulation is a clear aqueous solution for at least 2 hours at a temperature from about 0° C. to about 10° C. In some embodiments, the injectable pharmaceutical formulation is a clear aqueous solution for at least 3 hours at a temperature from about 0° C. to about 10° C. In some embodiments, the injectable pharmaceutical formulation is a clear aqueous solution for at least 6 hours at a temperature from about 0° C. to about 10° C. In some embodiments, the injectable pharmaceutical formulation is a clear aqueous solution for at least 8 hours at a temperature from about 0° C. to about 10° C. In some embodiments, the injectable pharmaceutical formulation is a clear aqueous solution for at least 24 hours at a temperature from about 0° C. to about 10° C.

In some embodiments, after a clear aqueous solution is filtered by a 0.22 micron filter, the amount of chlorambucil in the filtered aqueous solution is at least 96% of the total amount of chlorambucil in the aqueous solution before filtration. In some embodiments, after a clear aqueous solution is filtered by a 0.22 micron filter, the amount of chlorambucil in the filtered aqueous solution is at least 97% of the total amount of chlorambucil in the aqueous solution before filtration. In some embodiments, after a clear aqueous solution is filtered by a 0.22 micron filter, the amount of chlorambucil in the filtered aqueous solution is at least 98% of the total amount of chlorambucil in the aqueous solution before filtration. In some embodiments, after a clear aqueous solution is filtered by a 0.22 micron filter, the amount of chlorambucil in the filtered aqueous solution is at least 99% of the total amount of chlorambucil in the aqueous solution before filtration. In some embodiments, the aqueous formulation is free of solvent other than water. In some embodiments, the aqueous formulation is substantially free of solvent other than water.

In some embodiments, the injectable pharmaceutical formulation is free of a surfactant. In some embodiments, the surfactant is selected from CREMOPHOR® surfactants and Polysorbate 80.

In some embodiments, the injectable pharmaceutical formulation is substantially free of a surfactant. In some embodiments, the surfactant is selected from CREMOPHOR® surfactants and Polysorbate 80.

As used herein, the term “substantially free of surfactant” refers to a formulation containing less than 0.0005%, less than 0.0003%, or less than 0.0001% of surfactants and/or less than 0.0005%, less than 0.0003%, or less than 0.0001% of surfactant.

Also, provided herein is a method of treating a proliferative disease comprising the step of parenterally administering to a subject in need thereof of a therapeutically effective amount of a liquid pharmaceutical composition comprising the solid composition comprising the chlorambucil and the human serum albumin as described herein, and a pharmaceutically acceptable carrier.

As used herein, the terms “individual”, “patient”, or “subject” are used interchangeably and refer to any animal, including mammals, preferably mice, rats, other rodents, rabbits, dogs, cats, swine, cattle, sheep, horses, or primates, and most preferably humans.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “proliferative disease” refers to a disease caused by excessive proliferation of cells and turnover of cellular matrix. Non-limiting examples of proliferative diseases include cancer, atherosclerosis, arthritis (e.g. rheumatoid arthritis), psoriasis, fibrosis (e.g. pulmonary fibrosis, idiopathic pulmonary fibrosis), scleroderma and cirrhosis (e.g. cirrhosis of the liver).

Also, provided herein is a method of treating a cancer, the method comprising the step of parenterally administering to a subject in need thereof of a therapeutically effective amount of a liquid pharmaceutical composition comprising the solid composition comprising the chlorambucil and the human serum albumin as described herein, and a pharmaceutically acceptable carrier.

In some embodiments, the cancer is selected from the group consisting of bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

In some embodiments, cancer is selected from sarcoma, angiosarcoma, fibrosarcoma, rhabdomyosarcoma, liposarcoma, myxoma, rhabdomyoma, fibroma, lipoma, teratoma, non-small cell lung cancer (NSCLC), bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

As used herein, an “effective amount,” “therapeutically effective amount,” or a “pharmaceutically-effective amount” in reference to the compounds or compositions of the instant invention refers to the amount sufficient to induce a desired biological, pharmacological, or therapeutic outcome in a subject. That result can be reduction, prevention, mitigation, delay, shortening the time to resolution of, alleviation of the signs or symptoms of, or exert a medically-beneficial effect upon the underlying pathophysiology or pathogenesis of an expected or observed side-effect, toxicity, disorder or condition, or any other desired alteration of a biological system. In cancer treatment, the result will generally include the reduction, prevention, mitigation, limitation, and/or, delay of the deleterious physiological manifestations, growth or metastases of neoplasms.

As used herein, the term “preventing” means to completely or almost completely stop an disease or condition (e.g., cancer, metastatic cancer) from occurring, for example when the patient or subject is predisposed to an condition or is at risk of a disease or condition. Preventing can also include inhibiting, i.e., arresting the development, of a condition.

In some embodiments, the cancer is selected from the group consisting of a chronic lymphocytic leukemia, a Non Hodgkin's lymphoma, a Hodgkin's lymphoma, and a Waldenstrom macroglobulinaemia. In some embodiments, the cancer is a chronic lymphocytic leukemia. In some embodiments, the cancer is a Non Hodgkin's lymphoma. In some embodiments, the cancer is a Hodgkin's lymphoma. In some embodiments, the cancer is a Waldenstrom macroglobulinaemia.

In some embodiments, the method of treating cancer (e.g. any one of cancers described herein) comprises the step of parenterally administering to a subject in need thereof of a therapeutically effective amount of a liquid pharmaceutical composition comprising the solid composition comprising the chlorambucil and the human serum albumin as described herein, and a therapeutically effective amount of at least one inhibitor of the following kinases for the treatment of cancer: PIM, Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, flt-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf.

In some embodiments, the method of treating cancer (e.g. any one of cancers described herein) comprises the step of parenterally administering to a subject in need thereof of a therapeutically effective amount of a liquid pharmaceutical composition comprising the solid composition comprising the chlorambucil and the human serum albumin as described herein, and a therapeutically effective amount of at least one anti-cancer drug. Examples of an anti-cancer drug include aberaterone, aberaterone acetate, abarelix, aldesleukin, alemtuzumab, alitretinoin, allopurinol, altretamine, anastrozole, arsenic trioxide, asparaginase, azacitidine, bavituximab, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, enzalutamide, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

In some embodiments, a liquid pharmaceutical composition comprising the solid composition comprising the chlorambucil and the human serum albumin as described herein and an anti-cancer drug are administered simultaneously.

In some embodiments, a liquid pharmaceutical composition comprising the solid composition comprising the chlorambucil and the human serum albumin as described herein and an anti-cancer drug are administered consecutively.

The liquid pharmaceutical composition comprising the solid composition comprising the chlorambucil and the human serum albumin as described herein can be administered to an individual, such as human, via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. For example, the composition can be administered by inhalation to treat conditions of the respiratory tract. The composition can be used to treat respiratory conditions such as pulmonary fibrosis, broncheolitis obliterans, lung cancer, bronchoalveolar carcinoma, and the like. In some embodiments, the pharmaceutical composition is administrated intravenously.

The methods described herein may be performed alone or in conjunction with another therapy, such as surgery, radiation, chemotherapy, immunotherapy, gene therapy, and the like. Additionally, a person having a greater risk of developing the proliferative disease may receive treatments to inhibit or and/or delay the development of the disease.

As will be understood by those of ordinary skill in the art, the appropriate doses of chlorambucil will be approximately those already employed in clinical therapies wherein chlorambucil is administered alone or in combination with other chemotherapeutic agents.

In some embodiments, the amount of chlorambucil that is administered to a subject in need thereof with any one of solid pharmaceutical composition, aqueous composition, liquid pharmaceutical composition, or injectable pharmaceutical formulation as described herein is from about 0.01 mg/kg to about 10 mg/kg, from about 0.05 mg/kg to about 5 mg/kg, from about 0.05 mg/kg to about 4 mg/kg, from about 0.1 mg/kg to about 3 mg/kg, from about 0.5 mg/kg to about 3 mg/kg, from about 0.5 mg/kg to about 2.5 mg/kg, or from about 0.5 mg/kg to about 1 mg/kg. In some embodiments, the amount of chlorambucil that is administered to a subject in need thereof with any one of solid pharmaceutical composition, aqueous composition, liquid pharmaceutical composition, or injectable pharmaceutical formulation as described herein is about 0.01 mg/kg, about 0.02 mg/kg, about 0.03 mg/kg, about 0.05 mg/kg, about 0.1 mg/kg, about 0.2 mg/kg, about 0.3 mg/kg, about 0.4 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, or about 10 mg/kg. In some embodiments, any one of solid pharmaceutical composition, aqueous composition, liquid pharmaceutical composition, or injectable pharmaceutical formulation as described herein can be administered to a subject once daily, once weekly, or once biweekly. In some embodiments, any one of solid pharmaceutical composition, aqueous composition, liquid pharmaceutical composition, or injectable pharmaceutical formulation as described herein can be administered in dosage form as described herein (e.g., injection solution). Variation in dosage will likely occur depending on the condition being treated. Appropriate effective doses will also vary, as recognized by those skilled in the art, depending on the severity of the disease, the route of administration, the sex, age and general health condition of the subject, excipient usage, the possibility of co-usage with other therapeutic treatments such as use of other agents, and the judgment of the treating physician.

### Methods of Making

Also, provided herein are several methods to prepare an aqueous composition comprising chlorambucil and human serum albumin as described herein, or a solid composition comprising chlorambucil and human serum albumin as described herein.

Also, provided herein is a method of preparing a composition comprising chlorambucil and human serum albumin comprising the steps of:

(i) obtaining an organic solution of chlorambucil in polar water-miscible organic solvent;

(ii) obtaining a first aqueous solution comprising human serum albumin (HSA); and

(iii) mixing the organic solution comprising chlorambucil and the first aqueous solution comprising human serum albumin to obtain a second aqueous solution comprising the composition comprising chlorambucil and human serum albumin.

In some embodiments, the composition is a solid composition.

In some embodiments, the composition comprises a non-covalently bond complex comprising chlorambucil and the human serum albumin.

In some embodiments, the chlorambucil and the human serum albumin in the composition have a ratio by weight from about 1:20 to about 1:1000. In some embodiments, the chlorambucil and the human serum albumin in the composition have a ratio by weight from about 1:30 to about 1:800. In some embodiments, the chlorambucil and the human serum albumin in the composition have a ratio by weight from about 1:30 to about 1:600. In some embodiments, the chlorambucil and the human serum albumin in the composition have a ratio by weight from about 1:30 to about 1:250. In some embodiments, the chlorambucil and the human serum albumin in the composition have a ratio by weight from about 1:20 to about 1:120. In some embodiments, the chlorambucil and the human serum albumin in the composition have a ratio by weight from about 1:30 to about 1:110. In some embodiments, the chlorambucil and the human serum albumin in the aqueous composition have a ratio by weight from about 1:30 to about 1:120. In some embodiments, the chlorambucil and the human serum albumin in the aqueous composition have a ratio by weight from about 1:30 to about 1:150. In some embodiments, the chlorambucil and the human serum albumin in the composition have a ratio by weight from about 1:40 to about 1:150. In some embodiments, the chlorambucil and the human serum albumin in the composition have a ratio by weight from about 1:40 to about 1:120. In some embodiments, the chlorambucil and the human serum albumin in the composition have a ratio by weight of about 1:10, about 1:20, about 1:30, about 1:40, about 1:50, about 1:60, about 1:70, about 1:80, about 1:90, about 1:100, about 1:110, or about 1:120. In some embodiments, the chlorambucil and the human serum albumin in the composition have a ratio by weight of about 1:30, about 1:40, about 1:50, about 1:60, about 1:80, or about 110.

Non-limiting embodiments of these methods are as follows.

### Formation of the Organic Solution

As used herein, the term “organic solution” refers to a solution wherein at least one solvent is a non-aqueous solvent and the weight % of the non-aqueous solvent in the mixture of solvents is at least 50%, at least 60%, at least 70%, at least 90%, at least 95%, or at least 99%. In some embodiments, organic solution is a solution in which does not comprise water as a solvent.

In some embodiments, the terms “organic solvent” and “non-aqueous solvent” are used interchangeably and refer to a liquid comprising is at least 50%, at least 60%, at least 70%, at least 90%, at least 95%, or at least 99% of a solvent other than water. In some embodiments, the organic solvent does not comprise water.

In some embodiments, chlorambucil is dissolved in a polar organic solvent (e.g., an alcohol such as methanol, ethanol, isopropanol, and/or n-butanol; THF, CH₃CN; DMF; or mixtures thereof) to form an organic solution.

The polar organic solvent is miscible in water. In some embodiments, the polar organic solvent is an alcohol. In some embodiments, the polar organic solvent is ethanol or methanol, or mixtures thereof. For example, the polar organic solvent can be ethanol. In some embodiments, the polar organic solvent is methanol.

In some embodiments, the amount of polar organic solvent is from about 0.005 mL to about 5 mL per 1 mg of chlorambucil. In some embodiments, the amount of polar organic solvent is from about 0.01 mL to about 2 mL per 1 mg of chlorambucil. In some embodiments, the amount of polar organic solvent is from about 0.03 mL to about 1 mL per 1 mg of chlorambucil. In some embodiments, the amount of polar organic solvent is from about 0.03 mL to about 0.2 mL per 1 mg of chlorambucil. In some embodiments, the amount of polar organic solvent is from about 0.05 mL to about 1 mL per 1 mg of chlorambucil. In some embodiments, the amount of polar organic solvent is from about 0.1 mL to about 0.5 mL per 1 mg of chlorambucil. In some embodiments, the amount of the polar water-miscible organic solvent in the organic solution is from about 0.033 mL to about 0.125 mL per 1 mg of chlorambucil In some embodiments, the amount of organic solvent is about 0.01 mL, about 0.02 mL, about 0.03 mL, about 0.033 mL, about 0.04 mL, about 0.05 mL, about 0.06 mL, about 0.07 mL, about 0.08 mL, about 0.09 mL, about 0.1 mL, about 0.11 mL, about 0.12 mL, about 0.125 mL, about 0.13 mL, about 0.15 mL, about 0.2 mL, about 0.25 mL, about 0.5 mL, about 0.6 mL, about 0.7 mL, about 0.8 mL, about 0.9 mL, about 1.0 mL, about 2 mL, about 3 mL, about 4 mL, or about 5 mL per 1 mg of chlorambucil.

### Formation of the First Aqueous Solution

In some embodiments, a defined amount of human serum albumin is dissolved in an amount of aqueous solvent to form a first aqueous solution. In some aspects of these embodiments, the aqueous solvent is water.

In some embodiments, the amount of the aqueous solvent in the first aqueous solution is from about 0.005mL to about 10 mL per 1 mg of HSA. In some embodiments, the amount of the aqueous solvent in the first aqueous solution per 1 mg of HSA is from about 0.005mL to about 5 mL, about 0.01 mL to about 1 mL, from about 0.01 mL to about 0.5 mL, 0.01 mL to about 0.1 mL, 0.01 mL to about 0.05 mL, 0.015 mL to about 0.04 mL, or from about 0.015 mL to about 0.033 mL. In some embodiments, the amount of the aqueous solvent in the first aqueous solution per 1 mg of HSA is about 0.01 mL, about 0.015 mL, about 0.018 mL, about 0.02 mL, about 0.025 mL, about 0.03 mL, about 0.033 mL, or about 0.04 mL.In some embodiments, the resulting composition comprising the chlorambucil and the human serum albumin can have any ratio by weight of the chlorambucil to the human serum albumin as described herein. In some embodiments, the human serum albumin is a fatty acid free human serum albumin. In some embodiments, the human serum albumin is essentially fatty acid free.

In some embodiments, a commercially available solution of human serum albumin USP for infusion can be used to form a first aqueous solution with or without diluting with water.

In some embodiments, the preparation of the organic solution and the preparation of the first aqueous solution are performed concurrently.

In some embodiments, the preparation of the organic solution and the preparation of the first aqueous solution are performed sequentially. In some embodiments, the preparation of the organic solution is performed before the preparation of the first aqueous solution. In some embodiments, the preparation of the first aqueous solution is performed before the preparation of the organic solution.

### Formation of the Second Aqueous Solution

In some embodiments, the organic solution of chlorambucil is mixed with the first aqueous solution of human serum albumin to form a second aqueous solution. In some embodiments, the second aqueous solution is a clear aqueous solution.

In some embodiments, the volume ratio of the amount of water to the amount of the polar organic solvent is in a range from about 1:1 to about 1000:1. In some embodiments, the volume ratio of the amount of water to the amount of the polar organic solvent is in a range from about 1.5:1 to about 100:1. In some embodiments, the volume ratio of the amount of water to the amount of the polar organic solvent is in a range from about 2:1 to about 100:1. In some embodiments, the volume ratio of the amount of water to the amount of the polar organic solvent is in a range from about 4:1 to about 50:1. In some embodiments, the volume ratio of the amount of water to the amount of the polar organic solvent is in a range from about 6:1 to about 25:1. In some embodiments, the volume ratio of the amount of water to the amount of the polar organic solvent is about 1.5:1, about 2:1, about 2.2:1, about 2.3:1, about 2.4:1, about 2.5:1, about 3:1, about 4:1, about 5:1, about 6:1, about 7:1, about 8:1, about 9:1, about 10:1, about 15:1, about 15:1, about 18:1, about 20:1, about 25:1, about 30:1, about 35:1, about 40:1, about 45:1, about 50:1, about 55:1, about 60:1, about 65:1, about 70:1, about 75:1, about 80:1, about 90:1, about 100:1, about 200:1, about 300:1, about 400:1, about 500:1, about 600:1, about 700:1, about 800:1, about 900:1, or about 1000:1.

In some embodiments, the organic solution is added to the first aqueous solution to form a second aqueous solution. In some embodiments, the organic solution is added dropwise to the first aqueous solution to form a second aqueous solution. In some embodiments, the first aqueous solution is added to the organic solution to form a second aqueous solution. In some embodiments, the addition is dropwise. In some embodiments, the mixing is performed with agitation. In some embodiments, the mixing is performed with stirring. In some embodiments, the mixing is performed with shaking. In some embodiments, the mixing is carried out such that the Reynolds number in the resultant fluid forming the second aqueous solution is from about 1 to about 10000, from about 5 to about 9000, from about 10 to about 8000, from about 20 to about 7000, from about 30 to about 6000, from about 40 to about 5000, from about 40 to about 4000, from about 40 to about 3000, from about 40 to about 2000, from about 40 to about 1000, from about 1000 to about 10000, from about 2000 to about 9000, from about 2000 to about 8000, from about 2000 to about 7000, from about 2000 to about 6000, or from about 2000 to about 5000. In some embodiments, the mixing is carried out such that the Reynolds number in the resultant fluid of the second aqueous solution is about 20, about 40, about 100, about 500, about 1000, about 1500, about 2000, about 2500, about 3000, about 3500, about 4000, about 4500, about 5000, about 6000, about 7000, about 8000, or about 10000.

In some embodiments, the addition is done at the temperature from about 0° C. to about 30° C. In some embodiments, the addition is done at the temperature from about 0° C. to about 20° C. In some embodiments, the addition is done at the temperature from about 0° C. to about 10° C. In some embodiments, the addition is done at the temperature from about 0° C. to about 5° C. In some embodiments, the addition is done at the temperature about 0° C. In some embodiments, the addition is done at the temperature about 5° C. In some embodiments, the addition is done at the temperature about 10° C.

In some embodiments, the time of addition is in a range from about 0.1 min to about 24 hours. In some embodiments, the time of addition is in a range from about 1 min to about 2 hour. In some embodiments, the time of addition is in a range from about 1 min to about 1 hour. In some embodiments, the time of addition is in a range from about 5 min to about 30 min.

Removal of Organic Solvent and Water from the Second Aqueous Solution

In some embodiments, upon completion of mixing of the organic solution with the first aqueous solution to form the second aqueous solution, the polar organic solvent and water are removed from the second aqueous solution to provide a solid. In some embodiments, the solvents are removed by lyophilization. In some embodiments, the solvents are removed under a vacuum. In some embodiments, the solvents are removed using rotary evaporation.

In some embodiments, the second aqueous solution was filtered before removal of the solvents. For example, the second aqueous solution can be filtered by a 0.22 micron filter before removal of the solvents.

As used herein, the term “micron” refers to a unit of measure of one one-thousandth of a millimeter.

In some embodiments, the polar organic solvent is removed under reduced pressure. In some embodiments, the polar organic solvent is removed using rotary evaporation. In some embodiments, the polar organic solvent is removed under a vacuum. In some embodiments, the removal of the polar organic solvent yields a clear aqueous solution. In some embodiments, water is removed from the aqueous under a vacuum. In some embodiments, water is removed from the aqueous solution using rotary evaporation. In some embodiments, water is removed from the aqueous solution by lyophilization.

In some embodiments, the solvents including both water and organic solvent are removed from the second aqueous solution simultaneously to provide a solid composition. In some embodiments, the solvents are removed under a vacuum. In some embodiments, the solvents are removed using rotary evaporation. In some embodiments, the solvents are removed by lyophilization. In some embodiments, the second aqueous solution was filtered before removal of the solvents.

### Reconstitution of the Solid

In some embodiments, the solid comprising the chlorambucil and the human serum albumin is mixed with an aqueous solution. In some embodiments, the aqueous solution is a saline solution. In some embodiments, the aqueous solution is a 0.9% saline solution. In some embodiments, the aqueous solution is a 5% Dextrose solution. In some embodiments, the mixing is the addition of the aqueous solution to the solid. In some embodiments, the mixing is the addition of the solid to the aqueous solution. In some embodiments, the mixing reconstitutes the solid. In some embodiments, the mixing yields a clear aqueous solution.

Unless otherwise defined, all technical and scientific terms used herein have the same meaning as commonly understood by one of ordinary skill in the art to which this disclosure belongs. Methods and materials are described herein for use in the present disclosure; other suitable methods and materials known in the art can also be used. The materials, methods, and examples are illustrative only and not intended to be limiting. All publications, patent applications, patents, and other references mentioned herein are incorporated by reference in their entirety. In case of conflict, the present specification, including definitions, will control.

### Making Liquid Pharmaceutical Composition (Formulation)

In some embodiments, the present disclosure provides a method of making a liquid pharmaceutical composition comprising dissolving the solid pharmaceutical composition comprising chlorambucil and human serum albumin prepared as described herein in a pharmaceutically acceptable carrier. In some embodiments, the pharmaceutically acceptable carrier is saline. In some embodiments, the pharmaceutically acceptable carrier is 0.9% saline solution. In some embodiments, the liquid pharmaceutical composition is substantially free of solvent other than water. In some embodiments, the liquid pharmaceutical composition contains not more than about 1% of area percent of chlorambucil of the degradation product (I) as determined by HPLC or LCMS. In some embodiments, the liquid pharmaceutical composition contains not more than about 0.5% of area percent of chlorambucil of the degradation product (I). In some embodiments, the liquid pharmaceutical composition is a clear aqueous solution. In some embodiments, the liquid pharmaceutical composition is a clear aqueous solution for at least 1 hour. In some embodiments, the liquid pharmaceutical composition is a clear aqueous solution for at least 2 hours. In some embodiments, the liquid pharmaceutical composition is a clear aqueous solution for at least 6 hours at a temperature from about 0° C. to about 10° C. In some embodiments, the liquid pharmaceutical composition is free from a surfactant selected from CREMOPHOR® surfactants and Polysorbate 80. In some embodiments, the liquid pharmaceutical composition comprises a stabilizer selected from N-acetyltryptophanate and caprylic acid, or sodium salt thereof.

In some embodiments, the liquid pharmaceutical composition has pH value from about 5 to about 8. In some embodiments, the liquid pharmaceutical composition has pH value from about 5.5 to about 7.8. In some embodiments, the liquid pharmaceutical composition has pH value from about 6 to about 7.5. In some embodiments, the liquid pharmaceutical composition has pH value from about 6.5 to about 7.5. In some embodiments, the liquid pharmaceutical composition has pH value from about 6 to about 6.5. In some embodiments, the liquid pharmaceutical composition has pH value from about 6.5 to about 7. In some embodiments, the liquid pharmaceutical composition has pH value from about 7 to about 7.5. In some embodiments, the liquid pharmaceutical composition has pH value about 6, about 6.1, about 6.2, about 6.3, about 6.4, about 6.5, about 6.6, about 6.7, about 6.8, about 6.9, about 7.0, about 7.1, about 7.2, about 7.3, about 7.4, or about 7.5. In some embodiments, the liquid pharmaceutical composition is substantially free of solvent other than water. In some embodiments, the liquid pharmaceutical composition is free of solvent other than water.

In some embodiments, the liquid pharmaceutical composition is a clear aqueous solution, wherein the aqueous solution has pH value from about 5 to about 8, and wherein the aqueous solution is substantially free of solvent other than water. In some embodiments, the liquid pharmaceutical composition is a clear aqueous solution, wherein the aqueous solution has pH value from about 5 to about 8, and wherein the aqueous solution is free of solvent other than water. In some embodiments, the liquid pharmaceutical composition is a clear aqueous solution, wherein the aqueous solution has pH value from about 6 to about 7.5, and wherein the aqueous solution is substantially free of solvent other than water. In some embodiments, the liquid pharmaceutical composition is a clear aqueous solution, wherein the aqueous solution has pH value from about 6 to about 7.5, and wherein the aqueous solution is free of solvent other than water.

### EXAMPLES

### Materials and Methods

LCMS analysis: The LCMS system includes a HPLC system coupled to tandem mass spectrometry. The HPLC system used herein consists of a SHIMADZU LC-20AD pump, a SHIMADZU SIL-20AC auto sampler, a SHIMADZU SPD-M20A Diode Array Detector, and a SHIMADZU LCMS solution workstation. Agilent Zorbax XDB-C18 column (4.6mm×50mm, 5 μm) is used as an analytical HPLC column. Mobile phases A and B consist of water with 0.1% trifluoroacetic acid (TFA) and methanol with 0.1% TFA, respectively. The mobile phases were delivered at a programmed linear gradient. Separation was pumped at a flow rate of 0.6 ml/minute, and initiated and maintained at a mobile phase ratio of 85:15 (A:B) for 0.5 min. The ratio was changed to 10:90 (A:B) over a period of 1.5 min using a linear curve and then maintained at 10:90 (A:B) over a period of 4 min. The mobile phase was subsequently changed back to 85:15 (A:B) over a period of 1 min and this ratio was maintained for 1 min before the next sample was injected. The effluent is detected at a wavelength of 254nm using a UV detector. The sample injection amount is 10 μl.
HPLC analysis: The HPLC system used herein is a SHIMADZU LC-10AT vp series system, which consists of a SHIMADZU LC-10AT vp pump, a manual injector, a SHIMADZU CTO-10AS vp column oven, a SHIMADZU SPD-10A vp wavelength detector, and a SHIMADZU LC solution workstation. Waters XTERRA RP10 column (4.6mm×150 mm, 5 μm) is used as an analytical HPLC column. The column oven temperature is 30° C. Mobile phase is composed of methanol (added with 0.1% TFA) and water (70:30, v/v) and pumped at a flow rate of 1 ml/minute. The effluent is detected at a wavelength of 254 nm using a UV detector. The sample injection amount is 20 μl.

### Example 1

### Composition Comprising Chlorambucil and Human Serum Albumin (HSA)

The ratio by weight of chlorambucil to HSA prepared was about 1:110.

Chlorambucil (4 mg) was dissolved in methanol (0.5 ml) in a vial to give a clear solution. HSA (440 mg) (native fatty acid free human serum albumin purchased from SeraCare Life Sciences, product code: HS-455-80, which contains fatty acids<0.2 mg/gm) as a powder was dissolved in 8 ml of water in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

A sample of 100 mg of the lyophilized solid was reconstituted by adding 2 mL water to give a clear aqueous solution.

### Example 2

### Composition Comprising Chlorambucil and Human Serum Albumin (HSA)

The ratio by weight of chlorambucil to HSA prepared was about 1:80.

Chlorambucil (4 mg) was dissolved in methanol (0.5 ml) in a vial to give a clear solution. HSA (320 mg) (native fatty acid free human serum albumin purchased from SeraCare Life Sciences, product code: HS-455-80, which contains fatty acids <0.2 mg/gm) as a powder was dissolved in 8 ml of water in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

A sample of 100 mg of the lyophilized solid was reconstituted by adding 2 mL water to give a clear aqueous solution.

### Example 3

### Composition Comprising Chlorambucil and Human Serum Albumin (HSA)

The ratio by weight of chlorambucil to HSA prepared was about 1:110.

Chlorambucil (4 mg) was dissolved in methanol (0.5 ml) in a vial to give a clear solution. A solution of HSA (440 mg, 2.2 ml) (20% human serum albumin solution for infusion (product name: AlbuRx) from CSL Behring) was added into 5.8 ml of water to give a HSA solution (8 ml) in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

A sample of 100 mg of the lyophilized solid was reconstituted by adding 2 mL water to give a clear aqueous solution. This clear aqueous solution stays clear and is free of visible particles or precipitation when visually observed after 3 hours at room temperature. This clear aqueous solution stays clear and is free of visible particles or precipitation when visually observed after 6 hours at room temperature.

### Example 4

### Composition Comprising Chlorambucil and Human Serum Albumin (HSA)

The ratio by weight of chlorambucil to HSA prepared was about 1:80.

Chlorambucil (4 mg) was dissolved in methanol (0.5 ml) in a vial to give a clear solution. A solution of HSA (320 mg, 1.6 ml) (20% human serum albumin solution for infusion (product name: AlbuRx) from CSL Behring) was added into 6.4 ml of water to give a HSA solution (8 ml) in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

A sample of 100 mg of the lyophilized solid was reconstituted by adding 2 mL water to give a clear aqueous solution. This clear aqueous solution stays clear and is free of visible particles or precipitation when visually observed after 3 hours at room temperature. This clear aqueous solution stays clear and is free of visible particles or precipitation when visually observed after 6 hours at room temperature.

### Example 5

### Composition Comprising Chlorambucil and Human Serum Albumin (HSA)

The ratio by weight of chlorambucil to HSA prepared was about 1:60.

Chlorambucil (4 mg) was dissolved in methanol (0.5 ml) in a vial to give a clear solution. A solution of HSA (240 mg, 1.2 ml) (20% human serum albumin solution for infusion (product name: AlbuRx) from CSL Behring) was added into 6.8 ml of water to give a HSA solution (8 ml) in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

A sample of 100 mg of the lyophilized solid was reconstituted by adding 2 mL water to give a clear aqueous solution. This clear aqueous solution stays clear and is free of visible particles or precipitation when visually observed after 3 hours at room temperature. This clear aqueous solution stays clear and is free of visible particles or precipitation when visually observed after 6 hours at room temperature.

### Example 6

### Composition Comprising Chlorambucil and Human Serum Albumin (HSA)

The ratio by weight of chlorambucil to HSA prepared was about 1:50.

Chlorambucil (4 mg) was dissolved in methanol (0.5 ml) in a vial to give a clear solution. A solution of HSA (200 mg, 1 ml) (20% human serum albumin solution for infusion (product name: AlbuRx) from CSL Behring) was added into 2 ml of water to give a HSA solution (3 ml) in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

A sample of 100 mg of the lyophilized solid was reconstituted by adding 2 mL water to give a clear aqueous solution. This clear aqueous solution stays clear and is free of visible particles or precipitation when visually observed after 3 hours at room temperature. This clear aqueous solution stays clear and is free of visible particles or precipitation when visually observed after 6 hours at room temperature.

### Example 7

### Composition Comprising Chlorambucil and Human Serum Albumin (HSA)

The ratio by weight of chlorambucil to HSA prepared was about 1:50. Chlorambucil (20 mg) was dissolved in methanol (1 ml) in a vial to give a clear solution. A solution of HSA (1000 mg, 5 ml) (20% human serum albumin solution for infusion (product name: AlbuRx) from CSL Behring) was added into 20 ml of water to give a HSA solution (25 ml) in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

A sample of 100 mg of the lyophilized solid was reconstituted by adding 2 mL water to give a clear aqueous solution.

### Example 8

### Composition Comprising Chlorambucil and Human Serum Albumin (HSA)

The ratio by weight of chlorambucil to HSA prepared was about 1:40. Chlorambucil (40 mg) was dissolved in methanol (2 ml) in a vial to give a clear solution. A solution of HSA (1600 mg, 8 ml) (20% human serum albumin solution for infusion (product name: AlbuRx) from CSL Behring) was added into 32 ml of water to give a HSA solution (40 ml) in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

A sample of 100 mg of the lyophilized solid was reconstituted by adding 2 mL water to give a clear aqueous solution.

### Example 9

### Composition Comprising Chlorambucil and Human Serum Albumin (HSA)

The ratio by weight of chlorambucil to HSA prepared was about 1:30. Chlorambucil (6 mg) was dissolved in methanol (0.2 ml) in a vial to give a clear solution. A solution of HSA (180 mg, 0.9 ml) (20% human serum albumin solution for infusion (product name: AlbuRx) from CSL Behring) was added into 2.7 ml of water to give a HSA solution (3.6 ml) in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

A sample of 100 mg of the lyophilized solid was reconstituted by adding 2 mL water to give a slightly cloudy aqueous solution.

### Example 10

### Measure the Chlorambucil Stability in Aqueous Solution

0.05 ml of a methanol solution of chlorambucil (1 mg/ml) was added into a 0.95 ml water solution to give a clear solution. This chlorambucil aqueous solution was immediately injected into a LCMS system. The area percents of the peaks in the LCMS analysis related to chlorambucil, degradation product (I), and degradation product (II) were shown in the table 1. The same chlorambucil aqueous solution was also injected into the LCMS system at time points of 1 hour, 2 hour, 3 hour, 4 hour, and 5 hour. The area percents of the peaks in the

LCMS analysis related to chlorambucil, degradation product (I), and degradation product (II) at 1 hour, 2 hour, 3 hour, 4 hour, and 5 hour were also shown in the table 1.

TABLE 1

| | t (h) | | | | | |
|---|---|---|---|---|---|---|
| | 0 | 1 | 2 | 3 | 4 | 5 |
| Chlorambucil | 98.3 | 85.3 | 74.8 | 64.4 | 58.9 | 53.1 |
| (Area %) | | | | | | |
| Degradation product (I) | 1.7 | 14.7 | 25.1 | 35.2 | 40.5 | 46.5 |
| (Area %) | | | | | | |
| Degradation product (II) | 0 | 0 | 0.1 | 0.4 | 0.6 | 0.5 |
| (Area %) | | | | | | |

### Example 11

### Measure the Chlorambucil Stability at Room Temperature in Aqueous Solution of Composition Comprising Chlorambucil and Human Serum Albumin (HSA)

100 mg of the lyophilized solid from Example 1 (The ratio by weight of chlorambucil to HSA is about 1:110) was reconstituted by adding 2 mL water to give a clear aqueous solution. To 200 μl of the clear aqueous solution was added 800 μl of acetonitrile. The mixture was vortexed for seconds and then centrifuged at 4,000 g for 5 minutes. The supernatant was removed and collected followed by injection on LCMS system. The clear aqueous solution was kept at room temperature for 2 hours. The same procedure was repeated at the different time points of 0.5 hour, 1 hour, and 2 hour. The area percents of the peaks in the LCMS analysis related to Chlorambucil and degradation product (I) were shown in the table 2.

TABLE 2

| t (h) | 0 | 0.5 | 1 | 2 |
|---|---|---|---|---|
| Chlorambucil | 100 | 100 | 99.8 | 99.7 |
| (Area %) | | | | |
| Degradation product (I) | 0 | 0 | 0.2 | 0.3 |
| (Area %) | | | | |

### Example 12

### Measure the Chlorambucil Stability at Room Temperature in Aqueous Solution of Composition Comprising Chlorambucil and Human Serum Albumin (HSA)

100 mg of the lyophilized solid from Example 2 (The ratio by weight of chlorambucil to HSA is about 1:80) was reconstituted by adding 2 mL water to give a clear aqueous solution. To 200 μl of the clear aqueous solution was added 800 μl of acetonitrile. The mixture was vortexed for seconds and then centrifuged at 4,000 g for 5 minutes. The supernatant was removed and collected followed by injection on LCMS system. The clear aqueous solution was kept at room temperature for 2 hour. The same procedure was repeated at the different time points of 0.5 hour, 1 hour, and 2 hour. The area percents of the peaks in the LCMS analysis related to Chlorambucil and degradation product (I) were shown in the table 3.

TABLE 3

| t (h) | 0 | 0.5 | 1 | 2 |
|---|---|---|---|---|
| Chlorambucil | 100 | 100 | 99.7 | 99.6 |
| (Area %) | | | | |
| Degradation product (I) | 0 | 0 | 0.3 | 0.4 |
| (Area %) | | | | |

### Example 13

### Measure the Chlorambucil Stability at 0° C. in Aqueous Solution of Composition Comprising Chlorambucil and Human Serum Albumin (HSA)

100 mg of the lyophilized solid from Example 2 (The ratio by weight of chlorambucil to HSA is about 1:80) was reconstituted by adding 2 mL water to give a clear aqueous solution. The clear aqueous solution was kept at 0° C. for 2 hours. Immediately upon dissolving the lyophilized solid, to 200 μl of the clear aqueous solution was added 800 μl of acetonitrile. The mixture was vortexed for seconds and then centrifuged at 4,000 g for 5 minutes. The supernatant was removed and collected followed by injection on LCMS system. The same procedure was repeated at the different time points of 0.5 hour, 1 hour, and 2 hours. The area percents of the peaks in the LCMS analysis related to Chlorambucil and degradation product (I) were shown in the table 4.

TABLE 4

| t (h) | 0 | 0.5 | 1 | 2 |
|---|---|---|---|---|
| Chlorambucil | 100 | 100 | 100 | 100 |
| (Area %) | | | | |
| Degradation product (I) | 0 | 0 | 0 | 0 |
| (Area %) | | | | |

### Example 14

### Measure the Chlorambucil Stability at Room Temperature in Aqueous Solution of Composition Comprising Chlorambucil and Human Serum Albumin (HSA)

100 mg of the lyophilized solid from Example 3 (The ratio by weight of chlorambucil to HSA is about 1:110) was reconstituted by adding 2 mL water to give a clear aqueous solution. The clear aqueous solution was kept at room temperature for 7 hours. At 1 hour, to 200 μl of the clear aqueous solution was added 800 μl of acetonitrile. The mixture was vortexed for seconds and then centrifuged at 4,000 g for 5 minutes. The supernatant was removed and collected followed by injection on LCMS system. The same procedure was repeated at the different time points of 2 hours, 3 hours, 4 hours, 5 hours, 6 hours, and 7 hours. The area percents of the peaks in the LCMS analysis related to chlorambucil and degradation product (I) were shown in the table 5.

TABLE 5

| | t (h) | | | | | | |
|---|---|---|---|---|---|---|---|
| | 1 | 2 | 3 | 4 | 5 | 6 | 7 |
| Chlorambucil | 100 | 100 | 100 | 100 | 100 | 100 | 99.9 |
| (Area %) | | | | | | | |
| Degradation product | 0 | 0 | 0 | 0 | 0 | 0 | 0.1 |
| (I) (Area %) | | | | | | | |

### Example 15

### Measure the Chlorambucil Stability at Room Temperature in Aqueous Solution of Composition Comprising Chlorambucil and Human Serum Albumin (HSA)

100 mg of the lyophilized solid from Example 4 (The ratio by weight of chlorambucil to HSA is about 1:80) was reconstituted by adding 2 mL water to give a clear aqueous solution. The clear aqueous solution was kept at room temperature for 7 hours. At 1 hour, to 200 μl of the clear aqueous solution was added 800 μl of acetonitrile. The mixture was vortexed for seconds and then centrifuged at 4,000 g for 5 minutes. The supernatant was removed and collected followed by injection on LCMS system. The same procedure was repeated at the different time points of 2 hours, 3 hours, 4 hours, 5 hours, 6 hours, and 7 hours. The area percents of the peaks in the LCMS analysis related to Chlorambucil and degradation product (I) were shown in the table 6.

TABLE 6

| | t (h) | | | | | | |
|---|---|---|---|---|---|---|---|
| | 1 | 2 | 3 | 4 | 5 | 6 | 7 |
| Chlorambucil | 100 | 100 | 100 | 100 | 99.9 | 99.9 | 99.8 |
| (Area %) | | | | | | | |
| Degradation product | 0 | 0 | 0 | 0 | 0.1 | 0.1 | 0.2 |
| (I) (Area %) | | | | | | | |

### Example 16

### Measure the Chlorambucil Stability at Room Temperature in Aqueous Solution of Composition Comprising Chlorambucil and Human Serum Albumin (HSA)

100 mg of the lyophilized solid from Example 7 (The ratio by weight of chlorambucil to HSA is about 1:50) was reconstituted by adding 2 mL water to give a clear aqueous solution. The clear aqueous solution was kept at room temperature for 24 hours. At 0 hour, to 200 μlof the clear aqueous solution was added 800 μl of acetonitrile. The mixture was vortexed for seconds and then centrifuged at 4,000 g for 5 minutes. The supernatant was removed and collected followed by injection on LCMS system. The same procedure was repeated at the different time points of 1 hour, 2 hours, 3 hours, 4 hours, 5 hours, and 24 hours. The area percents of the peaks in the LCMS analysis related to Chlorambucil and degradation product (I) were shown in the table 7.

TABLE 7

| | t (h) | | | | | | |
|---|---|---|---|---|---|---|---|
| | 0 | 1 | 2 | 3 | 4 | 5 | 24 |
| Chlorambucil | 100 | 100 | 100 | 100 | 99.6 | 99.5 | 99.0 |
| (Area %) | | | | | | | |
| Degradation product | 0 | 0 | 0 | 0 | 0.4 | 0.5 | 1.0 |
| (I) (Area %) | | | | | | | |

### Example 17

### Measure the Correlation Between HPLC Peak Area and the Chlorambucil Concentration

Methanol solutions of chlorambucil in 6 different concentrations, 0.01 mg/ml, 0.025 mg/ml, 0.0375 mg/ml, 0.05 mg/ml, 0.075 mg/ml, and 0.1 mg/ml, were prepared. The 6 chlorambucil methanol solutions were analyzed in HPLC. The peak area and concentration of chlorambucil were correlated using linear regression. The linear regression data is shown as below.

Y(peak area)=43002+8.0065E7*X(concentration), R=0.99996, P<0.0001.

### Example 18

### Measure the Chlorambucil Concentrations in the Clear Aqueous Solutions Before and After the Filtration at 0 hour, and After the Filtration at 1 hour, and 2 hours

900 mg of the lyophilized solid of the composition comprising chlorambucil and HSA from Example 8 (The ratio by weight of chlorambucil to HSA is about 1:40) was dissolved in 18 ml of water to give a clear aqueous solution, which was kept at about 20° C. Immediately after the lyophilized solid was dissolved in water, 6 ml of the clear aqueous solution was taken out from the 18 ml solution. Then 1 ml of the solution was taken out from the 6 ml clear aqueous solution to give the solution CB-0-0 h, and the remaining 5 ml of the solution was filtered by the same 0.22 micron aqueous phase filter at 1 ml at a time to give the solutions CB-1-0 h, CB-2-0 h, CB-3-0 h, CB-4-0 h, and CB-5-0 h. To 200 μl of the solutions CB-0-0 h and CB-5-0 h were added 800 μl of acetonitrile separately. The mixtures were vortexed for seconds and then centrifuged at 4,000 g for 5 minutes. The supernatants were removed and collected followed by injection on HPLC. The same procedure was repeated 2 more times for each of solutions CB-0-0 h and CB-5-0 h. Based on the HPLC data and the measurement data of Example 17, the chlorambucil concentrations of the solutions of CB-0-0 h, and CB-5-0 h have been calculated and shown in the Table 8. At 0 hour, the chlorambucil concentration of the clear aqueous solution after the filtration was about 99.4% of the chlorambucil concentration of the clear aqueous solution before the filtration.

TABLE 8

| Solution | chlorambucil | Average chlorambucil |
|---|---|---|
| Number | Concentration (mg/ml) | Concentration (mg/ml) |
| CB-0-0h-1 | 1.016 | 1.017 |
| CB-0-0h-2 | 1.017 | |
| CB-0-0h-3 | 1.019 | |
| CB-5-0h-1 | 1.011 | 1.011 |
| CB-5-0h-2 | 1.011 | |
| CB-5-0h-3 | 1.012 | |

At 1 hour, 5 ml of the clear aqueous solution was taken out from the remaining 12 ml of the aqueous solution. Then 1 ml of the solution was taken out from the 5 ml clear aqueous solution and filtered by a 0.22 micron aqueous phase filter to give the solution CB-1-1 h, and the remaining 4 ml of the solution was filtered by the same 0.22 micron aqueous phase filter at 1 ml at a time to give the solutions CB-2-1 h, CB-3-1 h, CB-4-1 h, and CB-5-1 h. To 200 μl of the solution CB-5-1 h was added 800 μl of acetonitrile. The mixture was vortexed for seconds and then centrifuged at 4,000 g for 5 minutes. The supernatant was removed and collected followed by injection on HPLC. The same procedure was repeated 2 more times for the solution CB-5-1 h. Based on the HPLC data and the measurement data of Example 17, the chlorambucil concentrations of the solution CB-5-1 h have been calculated and shown in the Table 9. At 1 hour, the chlorambucil concentration of the clear aqueous solution after the filtration was about 98.4% of the chlorambucil concentration of the clear aqueous solution at 0 hour before the filtration.

TABLE 9

| Solution | chlorambucil | Average chlorambucil |
|---|---|---|
| Number | Concentration (mg/ml) | Concentration (mg/ml) |
| CB-5-1h-1 | 1.003 | 1.001 |
| CB-5-1h-2 | 1.001 | |
| CB-5-1h-3 | 1.000 | |

At 2 hour, 5 ml of the clear aqueous solution was taken out from the remaining 7 ml of the aqueous solution. Then 1 ml of the solution was taken out from the 5m1 clear aqueous solution and filtered by a 0.22 micron aqueous phase filter to give the solution CB-1-2 h, and the remaining 4 ml of the solution was filtered by the same 0.22 micron aqueous phase filter at 1 ml at a time to give the solutions CB-2-2 h, CB-3-2 h, CB-4-2 h, and CB-5-2 h. To 200 μl of the solution CB-5-2 h was added 800 μl of acetonitrile. The mixture was vortexed for seconds and then centrifuged at 4,000 g for 5 minutes. The supernatant was removed and collected followed by injection on HPLC. The same procedure was repeated 2 more times for the solution CB-5-2 h. Based on the HPLC data and the measurement data of Example, the chlorambucil concentrations of the solution CB-5-2 h have been calculated and shown in the Table 10. At 2 hour, the chlorambucil concentration of the clear aqueous solution after the filtration was about 97.3% of the chlorambucil concentration of the clear aqueous solution at 0 hour before the filtration.

TABLE 10

| Solution | chlorambucil | Average chlorambucil |
|---|---|---|
| Number | Concentration (mg/ml) | Concentration (mg/ml) |
| CB-5-2h-1 | 0.9934 | 0.9896 |
| CB-5-2h-2 | 0.9907 | |
| CB-5-2h-3 | 0.9847 | |

### Example 19

### Composition Comprising Chlorambucil and Human Serum Albumin (HSA)

The ratio by weight of chlorambucil to HSA prepared was about 1:40. Chlorambucil (30 mg) was dissolved in methanol (1.5 ml) in a vial to give a clear solution. A solution of HSA (1200 mg, 6 ml) (20% human serum albumin solution for infusion (product name: AlbuRx) from CSL Behring) was added into 6 ml of water to give a HSA solution (12 ml) in a round bottom flask. The methanol solution of chlorambucil was added slowly dropwise into the flask of the HSA solution with stirring at 0° C. Upon completion of the addition, a clear solution was obtained. The resulting clear aqueous solution was lyophilized overnight to give a white solid.

### Example 20

### Measure pH Value of the Clear Aqueous Solution of Composition Comprising Chlorambucil and Human Serum Albumin (HSA)

400 mg of the lyophilized solid of the composition comprising chlorambucil and HSA (the ratio by weight about 1:40) from Example 19 was dissolved in 10 ml of 0.9% saline solution, which had pH value about 5.41, to give a clear aqueous solution. The clear aqueous solution was kept at about 25° C. and measured for pH value. The pH value of the clear aqueous solution is 6.46 (3 measurements: 6.46, 6.46, and 6.45).

400 mg of the lyophilized solid of the composition comprising chlorambucil and HSA (the ratio by weight about 1:40) from Example 19 was dissolved in 10 ml of 5% Dextrose solution, which had pH value about 4.40, to give a clear aqueous solution. The clear aqueous solution was kept at about 25° C. and measured for pH value. The pH value of the clear aqueous solution is 6.49 (3 measurements: 6.48, 6.50, and 6.50).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-66. (canceled)

67. An aqueous composition comprising chlorambucil and human serum albumin, wherein the chlorambucil and the human serum albumin in the aqueous composition have a ratio by weight from about 1:20 to about 1:1000.

68. The aqueous composition of claim 67, wherein the chlorambucil and the human serum albumin in the aqueous composition have a ratio by weight from about 1:30 to about 1:600.

69. The aqueous composition of claim 67, wherein the chlorambucil and the human serum albumin in the aqueous composition have a ratio by weight from about 1:30 to about 1:150.

70. The aqueous composition of claim 67, wherein the human serum albumin is an aqueous solution of human serum albumin comprising at least one stabilizer, wherein the stabilizer is N-acetyltryptophanate or its sodium salt, or caprylic acid or its sodium salt.

71. The aqueous composition of claim 67, wherein the human serum albumin is an aqueous solution of human serum albumin comprising at least one stabilizer, wherein the stabilizer is caprylic acid or its sodium salt.

72. The aqueous composition of claim 67, wherein the composition is substantially free of solvent other than water.

73. The aqueous composition of claim 67, wherein the aqueous composition is a clear aqueous solution.

74. The aqueous composition of claim 73, wherein the aqueous composition is a clear aqueous solution for at least 1 hour .

75. The aqueous composition of claim 73, wherein the aqueous composition is a clear aqueous solution for at least 3 hours.

76. The aqueous composition of claim 67, wherein the aqueous composition has pH value from about 5 to about 8.

77. The aqueous composition of claim 67, wherein the aqueous composition has pH value from about 6 to about 7.5.

78. A solid composition produced by lyophilization of the aqueous composition of claim 67.

79. A liquid pharmaceutical composition comprising the solid composition of claim 78, and a pharmaceutically acceptable carrier.

80. The liquid pharmaceutical composition of claim 79, wherein the composition contains not more than about 1%, determined by area percent of chlorambucil, of the degradation product (I):

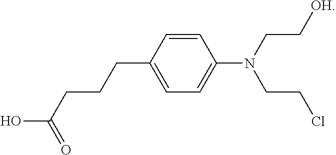 [SMILES: O=C(O)CCCc1ccc(N(CCO)CCCl)cc1]

81. The liquid pharmaceutical composition of claim 80, wherein the composition contains not more than about 0.1%, determined by area percent of chlorambucil, of the degradation product (I).

82. A method of treating a cancer, the method comprising a step of administering to a subject in need thereof a therapeutically effective amount of a liquid pharmaceutical composition of claim 79.

83. The method of claim 82, wherein the cancer is selected from the group consisting of a chronic lymphocytic leukemia, a Non Hodgkin's lymphoma, a Hodgkin's lymphoma, and a Waldenstrom macroglobulinaemia.

84. The method of claim 82, wherein the cancer is a chronic lymphocytic leukemia.
